(19) United States
(12) Patent Application Publication
(10) Pub. No.: US 2025/0236634 A1
(43) Pub. Date: Jul. 24, 2025

(54) ALKYL POLYGLYCOSIDE DERIVATIVES

(71) Applicant: Saudi Arabian Oil Company, Dhahran (SA)

(72) Inventors: Shaohua Chen, Beijing (CN); Jinxun Wang, Dhahran (SA); Tianping Huang, Beijing (CN); Mohammed Badri AlOtaibi, Dhahran (SA)

(21) Appl. No.: 18/416,595

(22) Filed: Jan. 18, 2024

(51) Int. Cl. C07H 15/04; C07D 487/04; C09K 8/58

(52) U.S. Cl. CPC C07H 15/04; C07D 487/04; C09K 8/58

(57) ABSTRACT

The present disclosure relates to alkyl polyglycoside (APG) carbonate salts, and to methods for viscosity reduction and displacement of heavy oils therewith. An exemplary embodiment includes an APG carbonate salt of Formula I:

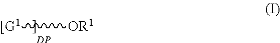

wherein R¹ is alkyl, G¹ is a glycose unit, and DP is the degree of polymerization of the APG carbonate salt.

### TECHNICAL FIELD

The present disclosure relates to alkyl polyglycoside derivatives, to methods of forming such, and to methods for viscosity reduction and displacement of heavy oils therewith.

### BACKGROUND

Extraction of hydrocarbons from subterranean formations involves the transport of hydrocarbons to the surface of the formation through a wellbore. However, extraction of heavy oils, which have a high viscosity, can be difficult. For example, the effectiveness of conventional displacement methods such as liquid flooding can be limited, due to the large mobility ratio between the displacing liquid (such as high-salinity water) and heavy oil.

Typical approaches to recovering heavy oils include lowering the viscosity of the oil, for example using heat and/or solvents, to make displacement and transport of the oil easier. However, such heat-based methods can be costly, while such solvent-based methods can have limited effectiveness, for example, where miscibility of the solvent and heavy oil is poor.

There accordingly remains a need for improved compositions and methods for viscosity reduction of heavy oils and/or displacement of heavy oils, for example, from subterranean formations.

### SUMMARY

Provided in the present disclosure is an alkyl polyglycoside (APG) carbonate salt of Formula I:

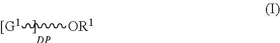

wherein:
R¹ is C₄₋₂₄ alkyl optionally substituted with 1-4 R^{A};
each R^{A} is independently halo, hydroxyl, —CN, C₁₋₆ alkenyl, C₂₋₆ alkynyl, C₃₋₈ cycloalkyl, C₆₋₁₀ aryl, 4- to 10-membered heterocycloalkyl, 5- to 10-membered heteroaryl, —NH₂, —NH(C₁₋₆ alkyl), —N(C₁₋₆ alkyl)₂, —O(C₁₋₆ alkyl), or —O(C₃₋₈ cycloalkyl);
G¹ is a glycose unit having Formula II:

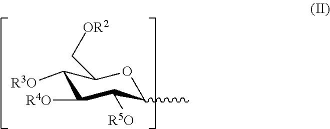

wherein:
   each R² and R³ is independently —H, -G¹, or

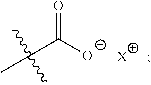

each R⁴ and R⁵ is independently —H or

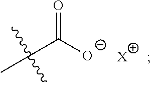

each X⁺ is independently a conjugate acid of a superbase; and
at least one instance of R², R³, R⁴, and R⁵ is

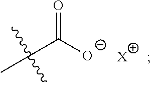

and
a degree of polymerization (DP) of the APG carbonate salt is from 1 to about 7.

Also provided in the present disclosure is a method of forming an APG carbonate salt, including reacting an APG with CO₂ in the presence of a superbase, in a solvent.

### BRIEF DESCRIPTION OF DRAWINGS

FIG. 1 is set of FT-IR spectra of a reaction mixture of the present disclosure, before and after introduction of CO₂.

FIG. 2 is a set of UV-vis spectra of a reaction mixture of the present disclosure, before and after introduction of CO₂.

FIG. 3 is a photograph of aqueous solutions of an alkyl polyglycoside (APG) carbonate salt of the present disclosure, at 25° C. and 90° C.

FIG. 4 is a graph showing the viscosity of a heavy crude oil as a function of shear rate, at temperatures of 30-90° C.

FIG. 5 is a graph showing the viscosity vs. shear rate of heavy oil and emulsions of the heavy oil and an aqueous solution of an APG carbonate salt of the present disclosure, at temperatures of 40-60° C.

FIG. 6 is an image of a micromodel of the present disclosure after heavy oil saturation.

FIG. 7 is an image of a micromodel of the present disclosure after injection of 2 PV of high-salinity water (HSW). The arrow indicates the flow direction of the injection fluid.

FIG. 8 is an image of a micromodel of the present disclosure after injection of 1 PV of an aqueous solution of an APG carbonate salt of the present disclosure. The arrow indicates the flow direction of the injection fluid.

FIG. 9 is an image of a micromodel of the present disclosure after further injection of HSW. The arrow indicates the flow direction of the injection fluid.

### DETAILED DESCRIPTION

The present disclosure relates to alkyl polyglycoside (APG) carbonate salts, to methods for preparing such salts, and to methods of viscosity reduction and displacement of heavy oil with such salts. The APG carbonate salts of the present disclosure include an anionic group including an APG carbonate, and a cationic group including a conjugate acid of a superbase. Such salts can be formed, for example, by reacting an APG with CO₂ in the presence of a superbase. Aqueous solutions of the APG carbonate salts of the present disclosure and a heavy oil can form emulsions having reduced viscosity as compared to the heavy oil alone, which are accordingly easier to displace and transport as compared to the heavy oil alone. Such emulsions can be easier to displace, for example, through a wellbore of a subterranean formation. Accordingly, the APG carbonate salts of the present disclosure can provide improved oil displacement in chemical flooding enhanced oil recovery (EOR) methods. Additionally, because CO₂ is reacted to form the APG carbonate salts, the methods described in the present disclosure can reduce atmospheric CO₂.

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

### Definitions

The terms “a,” “an,” and “the” are used in the present disclosure to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed in this disclosure, and not otherwise defined, is for the purpose of description only and not of limitation. Any use of section headings is intended to aid reading of the document and is not to be interpreted as limiting; information that is relevant to a section heading may occur within or outside of that particular section.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (for example, 1%, 2%, 3%, and 4%) and the sub-ranges (for example, 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

As used in the present disclosure, the term “about” can allow for a degree of variability in a value or range, for example, within 10%, within 5%, or within 1% of a stated value or of a stated limit of a range.

In the methods described in the present disclosure, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “substituted” means that an atom or group of atoms formally replaces hydrogen as a “substituent” attached to another group. The term “substituted”, unless otherwise indicated, refers to any level of substitution, for example, mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, for example, oxo, can replace two hydrogen atoms.

As used in the present disclosure, the term “Cₙ₋ₘ” indicates a range which includes the endpoints, wherein n and m are integers and indicate the number of carbons. Examples include C₁₋₄, C₁₋₆ and the like.

As used in the present disclosure, the term “n-membered,” where n is an integer, indicates the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

As used in the present disclosure, the term “alkyl” refers to a saturated hydrocarbon group that may be straight-chained or branched. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like.

As used in the present disclosure, the term “alkenyl,” refers to a straight-chain or branched hydrocarbon group corresponding to an alkyl group having one or more double carbon-carbon bonds. Example alkenyl groups include, but are not limited to, ethenyl, n-propenyl, isopropenyl, n-butenyl, sec-butenyl and the like.

As used in the present disclosure, the term “alkynyl” refers to either a straight chain or branched hydrocarbon corresponding to an alkyl group having one or more triple carbon-carbon bonds. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, 1,3,5-hexatriynyl and the like.

As used in the present disclosure, the term “cycloalkyl” refers to a non-aromatic hydrocarbon ring system (monocyclic, bicyclic or polycyclic), including cyclized alkyl and alkenyl groups. Cycloalkyl groups can include mono- or polycyclic (for example, having 2, 3 or 4 fused rings) groups and spirocycles. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (having a bond in common with) to the cycloalkyl ring, for example, benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like.

As used in the present disclosure, the term “Cₙ₋ₘ aryl” refers to an aryl group having from n to m ring carbon atoms. Aryl groups include phenyl, naphthyl, indanyl, indenyl, and the like.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from nitrogen, sulfur oxygen and phosphorus. Heterocycloalkyl groups can include mono- or bicyclic (for example, having two fused or bridged rings) ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (for example, C(O), S(O), C(S), S(O)₂, N-oxide) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (having a bond in common with) to the heterocycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include azetidinyl, azepanyl, dihydrobenzofuranyl, dihydrofuranyl, dihydropyranyl, morpholino, 3-oxa-9-azaspiro[5.5]undecanyl, 1-oxa-8-azaspiro[4.5]decanyl, piperidinyl, piperazinyl, oxopiperazinyl, pyranyl, pyrrolidinyl, quinuclidinyl, tetrahydrofuranyl, tetrahydropyranyl, 1,2,3,4-tetrahydroquinolinyl, tropanyl, and thiomorpholino.

As used in the present disclosure, the term “heteroaryl” refers to a monocyclic or polycyclic aromatic heterocycle having at least one heteroatom ring member selected from sulfur, oxygen and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. Example heteroaryl groups include, but are not limited to, pyridinyl (pyridyl), pyrimidinyl, pyrazinyl, pyridazinyl, pyrrolyl, pyrazolyl, azolyl, oxazolyl, thiazolyl, imidazolyl, furanyl, thiophenyl, quinolinyl, isoquinolinyl, naphthyridinyl (including 1,2-, 1,3-, 1,4-, 1,5-, 1,6-, 1,7-, 1,8-, 2,3- and 2,6-naphthyridine), indolyl, benzothiophenyl, benzofuranyl, benzisoxazolyl, imidazo[1,2-b]thiazolyl, purinyl, and the like.

As used in the present disclosure, the term “halo” refers to —F, —Cl, —Br, or -.

As used in the present disclosure, the term “hydroxyl” refers to —OH.

As used in the present disclosure, the term “oxo” refers to =O.

As used in the present disclosure, the term “alkyl polyglycoside” refers to alkyl mono-, di-, tri-, and oilgoglycosides, and to any mixture thereof. Alkyl polyglycosides can be synthesized, for example, by direct reaction of a carbohydrate with a fatty alcohol, or by two-stage transacetalization, in which a carbohydrate is reacted with a short-chain alcohol (and optionally depolymerized) before transacetalization with a fatty alcohol. The number of glycose units per —O-alkyl group of an individual alkyl polyglycoside, or the average number of glycoside units per —O-alkyl group of a mixture of alkyl polyglycosides, can each be referred to as the “degree of polymerization” (DP) of the alkyl polyglycoside.

As used in the present disclosure, the term “superbase” refers to compounds having a very high basicity, such as 1,8-diazabicyclo[5.4.0]undec-7-ene (DBU). Superbases include any species with a higher absolute proton affinity (APA=245.3 kcal/mole) and intrinsic gas phase basicity (GB=239 kcal/mole) as compared to to 1,8-bis-(dimethylamino)-naphthalene.

As used in the present disclosure, the term “subterranean formation” refers to any material under the surface of the earth, including under the surface of the bottom of the ocean. For example, a subterranean formation or material can be any section of a wellbore and any section of a subterranean petroleum- or water-producing formation or region in fluid contact with the wellbore. Injecting a material in a subterranean formation can include contacting the material with any section of a wellbore or with any subterranean region in fluid contact therewith. Subterranean materials can include any materials placed into the wellbore such as cement, drill shafts, liners, tubing, casing, or screens; injecting a material in a subterranean formation can include contacting with such subterranean materials. In some examples, a subterranean formation or material can be any below-ground region that can produce liquid or gaseous petroleum materials, water, or any section below-ground in fluid contact therewith.

As used in the present disclosure, the term “heavy oil” refers to viscous oils that cannot easily flow from production wells under normal reservoir conditions. For example, “heavy crude oil” can include any liquid petroleum with an API gravity of less than 20°.

### Alkyl Polyglycoside Carbonate Salts

Provided in the present disclosure are alkyl polyglycoside (APG) carbonate salts of Formula I:

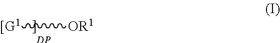

wherein R¹ is alkyl, G¹ is a glycose unit, and DP is the degree of polymerization of the APG carbonate salt.

R¹ of Formula I is C₄₋₂₄ alkyl optionally substituted with 1-4 R^{A}, and each R^{A} is independently halo, hydroxyl, —CN, C₁₋₆ alkenyl, C₂₋₆ alkynyl, C₃₋₈ cycloalkyl, C₆₋₁₀ aryl, 4- to 10-membered heterocycloalkyl, 5- to 10-membered heteroaryl, —NH₂, —NH(C₁₋₆ alkyl), —N(C₁₋₆ alkyl)₂, —O(C₁₋₆ alkyl), or —O(C₃₋₈ cycloalkyl). In some embodiments of Formula I, R¹ is branched alkyl. In some embodiments of Formula I, R¹ is straight-chained alkyl. In some embodiments of Formula I, R¹ is C₄₋₁₈ alkyl, C₄₋₁₆ alkyl, C₆₋₂₄ alkyl, C₆₋₁₈ alkyl, C₆₋₁₆ alkyl, C₈₋₂₄ alkyl, C₈₋₁₈ alkyl, or C₈₋₁₆ alkyl. In some embodiments of Formula I, R¹ is straight-chained C₆, C₈, C₁₀, C₁₂, C₁₄, C₁₆, or C₁₈ alkyl.

In some embodiments of Formula I, R¹ is substituted with 1-3, 1-2, 2-4, or 2-3 R^{A}. In some embodiments of Formula I, R¹ is substituted with 1 R^{A}. In some embodiments of Formula I, each R^{A} is independently halo, hydroxyl, or —CN. In some embodiments of Formula I, R¹ is unsubstituted.

G¹ of Formula I is a glycose unit of Formula II:

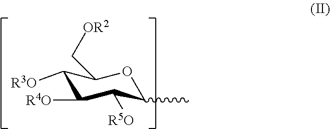

wherein at least one instance of R², R³, R⁴, and R⁵ is an ion pair including a carbonate anion:

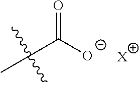

wherein X⁺ is a conjugate acid of a superbase.

Each R² and R³ of Formula II is independently —H, -G¹, or

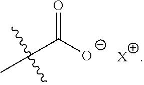

In some embodiments, DP of Formula I is 1, and each R² and R³ are independently —H or

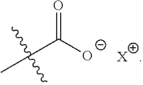

In some embodiments, DP of Formula I is greater than 1, and at least one instance of R² or R³ is -G¹.

In some embodiments, each R³ is independently —H or -G¹. In some embodiments, the APG carbonate salt of Formula I has Formula I-A:

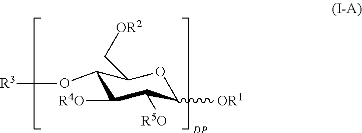

wherein each R², R³, R⁴, and R⁵ is independently —H or

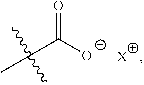

and at least one instance of R², R³, R⁴, or R⁵ is

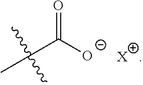

In some embodiments, DP of Formula I-A is from 1 to about 4.

In some embodiments, at least one instance of R² is O

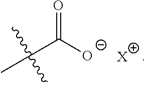

In some embodiments, one instance of R² is

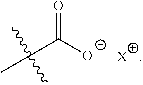

In some embodiments, the APG carbonate salt of Formula I has Formula I-B:

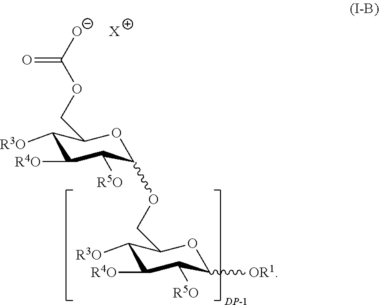

wherein (DP-1) is 1 less than the degree of polymerization of the APG carbonate salt. In some embodiments, (DP-1) of Formula I-B is from 0 to about 3.

In some embodiments, at least one instance of R³ is —H. In some embodiments, each R³ is —H. In some embodiments, at least one instance of R⁴ is —H. In some embodiments, each R⁴ is —H. In some embodiments, at least one instance of R⁵ is —H. In some embodiments, each R⁵ is —H.

DP of Formula I is from 1 to about 7. In some embodiments, DP of Formula I is from 1 to about 6, from 1 to about 5, from 1 to about 4, from 1 to about 3, from 1 to about 2, from 1 to about 1.8, from 1 to about 1.5, from about 1.1 to about 4, from about 1.1 to about 3, from about 1.1 to about 2, from about 1.1 to about 1.8, or from about 1.1 to about 1.5. In some embodiments, DP of Formula I is 1, or about 1.1, about 1.2, about 1.3, about 1.4, or about 1.5.

In some embodiments, each X⁺ is independently a conjugate acid of an organic superbase. In some embodiments, the conjugate acid of the organic superbase has a pKₐ of about 8 to about 13.5, measured in water.

In some embodiments, each X⁺ is independently a conjugate acid of a nitrogen-containing superbase, such as a phosphazene, amidine, guanidine, multicyclic polyamine, or any combination thereof. In some embodiments, each X⁺ is independently a conjugate acid of 1,8-Diazabicyclo[5.4.0]undec-7-ene (DBU), 1,5,7-Triazabicyclo[4.4.0]dec-5-ene (TBD), 1,4-Diazabicyclo[2.2.2]octane (DABCO), 1,1,3,3-Tetramethylguanidine (TMG), 1,5-Diazabicyclo[4.3.0]-5-nonene (DBN), or 7-Methyl-1,5,7-triazabicyclo[4.4.0]dec-5-ene (MTBD). In some embodiments, each X⁺ is a conjugate acid of DBU:

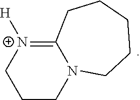 [SMILES: C1CCC2=[NH+]CCCN2CC1]

### Methods for Preparing APG Carbonate Salts

Also provided in the present disclosure are methods for preparing an APG carbonate salt. In some embodiments, the method includes reacting an APG with CO₂ in the presence of a superbase, in a solvent. In some embodiments, the method yields an APG carbonate salt of Formula I of the present disclosure. The methods of the present disclosure can be carried out under low CO₂ pressure and at low temperature, as compared to certain conventional carbonation methods.

In some embodiments, the method includes reacting an APG of Formula III:

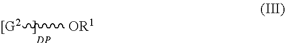

wherein R¹ is alkyl, G² is a glycose unit, and DP is the degree of polymerization of the APG.

R¹ of Formula III is C₄₋₂₄ alkyl optionally substituted with 1-4 R^{A}, and each R^{A} is independently halo, hydroxyl, —CN, C₁₋₆ alkenyl, C₂₋₆ alkynyl, C₃₋₈ cycloalkyl, C₆₋₁₀ aryl, 4- to 10-membered heterocycloalkyl, 5- to 10-membered heteroaryl, —NH₂, —NH(C₁₋₆ alkyl), —N(C₁₋₆ alkyl)₂, —O(C₁₋₆ alkyl), or —O(C₃₋₈ cycloalkyl). In some embodiments of Formula III, R¹ is straight-chained alkyl. In some embodiments of Formula III, R¹ is C₄₋₁₈ alkyl, C₄₋₁₆ alkyl, C₆₋₂₄ alkyl, C₆₋₁₈ alkyl, C₆₋₁₆ alkyl, C₈₋₂₄ alkyl, C₈₋₁₈ alkyl, or C₈₋₁₆ alkyl. In some embodiments of Formula III, R¹ is straight-chained C₆, C₈, C₁₀, C₁₂, C₁₄, C₁₆, or C₁₈ alkyl.

In some embodiments of Formula III, R¹ is substituted with 1-3, 1-2, 2-4, or 2-3 R^{A}. In some embodiments of Formula III, R¹ is substituted with 1 R^{A}. In some embodiments of Formula III, each R^{A} is independently halo, hydroxyl, or —CN. In some embodiments of Formula III, R¹ is unsubstituted.

G² of Formula III is a glycose unit of Formula IV:

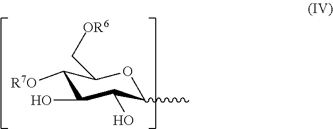

wherein each R⁶ and R⁷ is independently —H or -G².

In some embodiments, DP of Formula III is 1, and each R⁶ and R⁷ are —H. In some embodiments, DP of Formula III is greater than 1, and at least one instance of R⁶ and R⁷ is -G².

In some embodiments, each R⁶ is —H. In some embodiments, the APG of Formula III has Formula III-A:

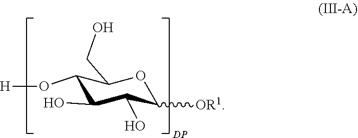

In some embodiments, DP of Formula III-A is from 1 to about 4.

In some embodiments of Formula III, each R⁷ is —H. In some embodiments, the APG of Formula III has Formula III-B:

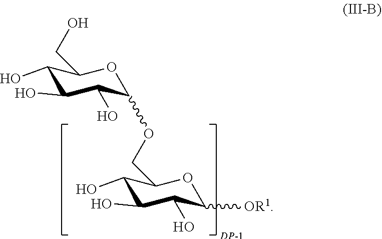

wherein (DP-1) is 1 less than the degree of polymerization of the APG. In some embodiments, (DP-1) of Formula III-B is from 0 to about 3.

DP of Formula III is from 1 to about 7. In some embodiments, DP of Formula III is from 1 to about 6, from 1 to about 5, from 1 to about 4, from 1 to about 3, from 1 to about 2, from 1 to about 1.8, from 1 to about 1.5, from about 1.1 to about 4, from about 1.1 to about 3, from about 1.1 to about 2, from about 1.1 to about 1.8, or from about 1.1 to about 1.5. In some embodiments, DP of Formula III is 1, or about 1.1, about 1.2, about 1.3, about 1.4, or about 1.5.

In some embodiments of the method, the superbase includes an organic superbase. In some embodiments, the superbase includes a nitrogen-containing compound, such as a phosphazene, amidine, guanidine, multicyclic polyamine, or any combination thereof. In some embodiments, the organic superbase includes DBU, TBD, DABCO, TMG, DBN, or MTBD. In some embodiments, the organic superbase includes DBU.

In some embodiments, the solvent includes dimethyl sulfoxide (DMSO). In some embodiments, reacting the APG with CO₂ includes contacting the APG with CO₂ gas, for example, at a pressure of less than about 5 bar, or about 3 to about 5 bar. In some embodiments, reacting the APG with CO₂ includes contacting the APG with CO₂ at a temperature of less than about 80° C., for example, about 50° C. to about 60° c. In some embodiments, reacting the APG is reacted with CO₂ in a high-temperature, high-pressure reactor.

In some embodiments, reacting the APG with CO₂ in the presence of a superbase yields a precipitate, and the method further includes separating the precipitate from the solvent, for example, by filtration or centrifugation. In some embodiments, separating the precipitate includes centrifuging, for example, at about 5,000 rpm.

Also provided in the present disclosure are APG carbonate salts prepared by a method of the present disclosure.

### Methods of Using APG Carbonate Salts

Also provided in the present disclosure are methods of using an APG carbonate salt of the present disclosure. For example, provided in the present disclosure is a method of reducing a viscosity of a heavy oil, the method including adding an aqueous solution of an APG carbonate salt of the present disclosure to the heavy oil. In another example, provided in the present disclosure is a method of displacing heavy oil, the method including injecting, through a wellbore into a subterranean formation including the heavy oil, an aqueous solution of an APG carbonate salt of the present disclosure. In some embodiments, the displacement method is an enhanced oil recovery method.

In some embodiments, the aqueous solution includes the APG carbonate salt in a concentration of about 0.01 wt % to about 5 wt %, for example, about 0.01 wt % to about 2 wt %, about 0.01 wt % to about 1 wt %, about 0.01 wt % to about 0.8 wt %, about 0.01 wt % to about 0.5 wt %, about 0.05 wt % to about 5 wt %, about 0.05 wt % to about 2 wt %, about 0.05 wt % to about 1 wt %, about 0.05 wt % to about 0.8 wt %, or about 0.05 wt % to about 0.5 wt %.

In some embodiments, the aqueous solution includes high-salinity water (HSW). For example, in some embodiments, the aqueous solution includes about 10,000 mg/L to about 100,000 mg/L of ions, for example, about 20,000 mg/L to about 80,000 mg/L, about 20,000 mg/L to about 65,000 mg/L, about 30,000 mg/L to about 100,000 mg/L, about 30,000 mg/L to about 80,000 mg/L, or about 30,000 mg/L to about 65,000 mg/L of ions. In some embodiments, the ions includes Na⁺, Ca²⁺, Mg²⁺, or any combination thereof. In some embodiments, the ions includes SO₄²⁻, Cl⁻, HCO₃⁻, or any combination thereof.

### EXAMPLES

### Example 1—Preparation of APG Carbonate Salt

5 g of an APG having an estimated molecular weight of about 600-800 g/mol and a 3-fold molar excess of DBU were mixed and then dissolved in 100 mL of DMSO. The solution was transferred into the reaction vessel of a high-temperature and high-pressure (HTHP) reactor. The glass vessel was then sealed, and the solution was stirred at a temperature of about 50-60° C. CO₂ was injected into the reactor from a gas cylinder to maintain a CO₂ environment at a pressure of about 3-5 bar. Over several hours, the APG carbonate salt precipitated from solution as a white solid. The reaction mixture was transferred to a centrifuge tube and centrifuged at 5,000 rpm for 10 min to recover the APG carbonate salt.

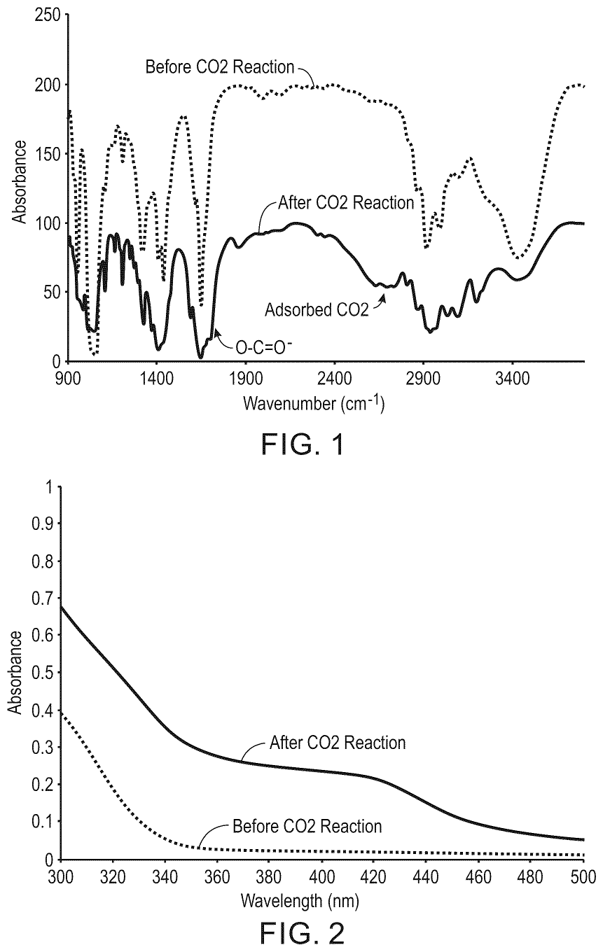

The reaction mixture was analyzed by Fourier-transform infrared (FTIR) and ultraviolet-visible (UV-vis) spectroscopy before and after introduction of CO₂. Results are shown in FIGS. 1 and 2, respectively. As indicated by arrows in FIG. 1, after introduction of CO₂, a band corresponding to dissolved CO₂ appeared, the peak at 1630 cm⁻¹ broadened, and a peak appeared at 1270 cm⁻¹, suggesting that hydroxyl groups of the APG were modified to —C(O)O— groups. Additionally, FIG. 2 shows an increase in absorbance after CO₂ introduction, indicating a change of the conjugated aromatic structure of DBU, due to formation of an ionic liquid (IL) including the conjugate acid of DBU and carbonate anion. The results accordingly suggested successful synthesis of the APG carbonate salt.

### Example 2—High-Salinity Water (HSW) Compatibility of APG Carbonate Salts

APG carbonate salt prepared according to Example 1 was added at a concentration of 0.2 wt % to high-salinity water (HSW) having a total dissolved solids (TDS) content of 57,670 mg/L (Table 1).

TABLE 1
Composition of HSW

| | Concentration |
|---|---|
| Ion | (mg/L) |
| Na⁺ | 18,300 |
| Ca²⁺ | 650 |
| Mg²⁺ | 2,110 |
| SO₄²⁻ | 4,290 |
| Cl⁻ | 32,200 |
| HCO₃⁻ | 120 |

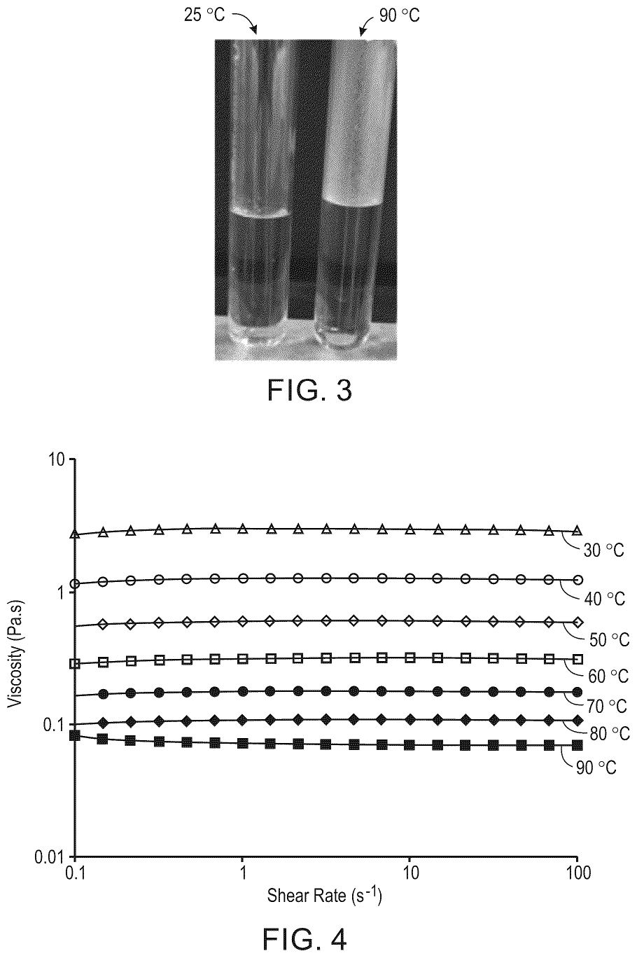

As shown in FIG. 3, the solution of APG carbonate salt in HSW remained transparent at 25° C. and 90° C., indicating excellent compatibility.

### Example 3—Viscosity Reduction of Heavy Oil

To investigate the viscosity reducing properties of the APG carbonate salt of Example 1, a heavy oil sample was first collected from a carbonate reservoir, degassed, and cleaned by centrifuge to remove sands and brine. The saturate, aromatic, resin and asphaltene (SARA) content of the sample was 36.2%, 27.2%, 25.5%, and 11.1%, respectively. The API gravity and density of the sample were 15.1° and 0.97 kg/m³, respectively. Prior to use, the heavy oil was stirred in a water bath at 70° C. for several hours to remove water and air bubbles. A plot of viscosity versus shear rate of the heavy oil at various temperature is shown in FIG. 4.

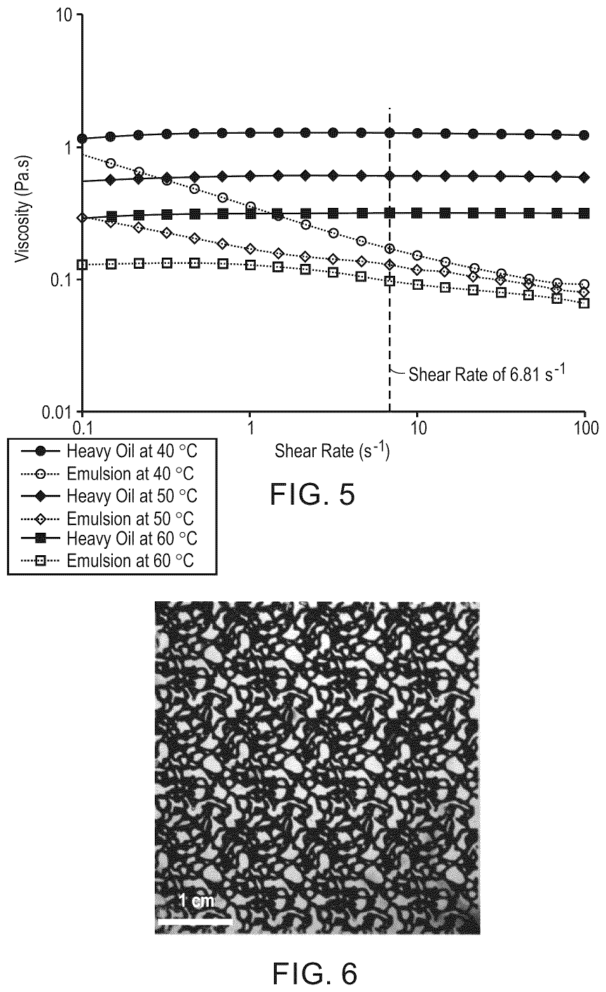

Emulsions of the heavy oil sample and water (7:3 oil-to-water ratio) including 0.2 wt % of the APG carbonate salt of Example 1 were prepared. FIG. 5 compares the viscosity of the heavy oil sample and the APG carbonate salt-containing emulsions at temperatures of 40-60° C. Viscosity reduction rates f_{VR} were calculated at various temperatures based on measured viscosity using the following equation.

$${f}_{VR} = \frac{{u}_{0} - {u}_{e}}{{u}_{0}} \times 100 \%$$

where u₀ and uₑ represent viscosities of dehydrated and degassed heavy crude oil and oil-water emulsion, respectively; measured values and results are listed in Table 2, below.

TABLE 2
Viscosity (at 6.81 s⁻¹) and f_{VR} of heavy
oil and APG carbonate salt-containing emulsions

| | Heavy oil | Emulsion | |
|---|---|---|---|
| T (° C.) | viscosity (Pa · s) | viscosity (Pa · s) | f_{VR} (%) |
| 40 | 1.26 | 0.169 | 87 |
| 50 | 0.60 | 0.129 | 79 |
| 60 | 0.31 | 0.095 | 70 |

As shown in FIG. 5 and Table 2, the APG carbonate salt exhibited excellent performance in heavy oil viscosity reduction.

### Example 4—Heavy Oil Displacement

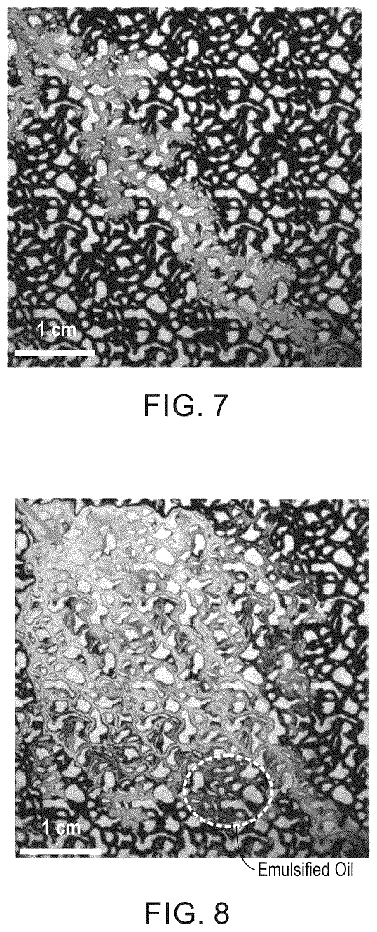

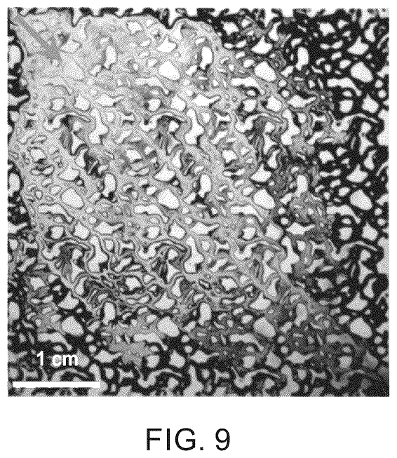

Heavy oil production by the flooding with an APG carbonate salt-containing solution at 50° C. was evaluated using micromodel equipment, by injecting a sequential set of slugs of varying pore volumes (PVs): (1) HSW injection for 2.0 PV, (2) APG carbonate salt solution (0.2 wt %) for 1.0 PV, and then (3) an extended HSW injection to maximize heavy oil production. Images of the micromodel after initial heavy oil saturation and after each injection slug are shown in FIGS. 6-9. A large amount of residual oil as bulky oil blocks was observed inside the micromodel after 2.0 PV injection of the HSW (FIG. 7). Oil production was not sensitive to the 2.0 PV HSW injection due to a large mobility ratio, as oil production remained nearly unchanged after a dominant flow channel was created from inlet to outlet of the micromodel. As shown in FIG. 8, after injection of the APG carbonate salt solution for 1.0 PV, heavy oil production increased markedly, as compared to the previous HSW injection. The micromodel observation demonstrated a strong interaction between the APG carbonate salt and heavy crude oil. A large amount of the residual oil blocks left after the HSW flooding were replaced by widespread emulsion due to the strong emulsification of heavy oil and the APG carbonate salt solution. The viscosity of the heavy oil was significantly decreased, resulting in a relatively large volumetric sweep efficiency. The dominant flow channel formed by water flooding was not observed, because the APG carbonate salt solution injection altered the mobility ratio of water and heavy oil. Subsequent water flooding had little to no effect on heavy oil production (FIG. 9), as the viscosity of HSW was too low to displace more emulsified heavy oil. The micromodel results demonstrate that the APG carbonate salt was effective in heavy oil displacement.

### EMBODIMENTS

Certain embodiments of the present disclosure are provided in the following list:

Embodiment 1. An alkyl polyglycoside (APG) carbonate salt of Formula I:

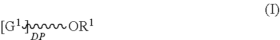

wherein:
R¹ is C₄₋₂₄ alkyl optionally substituted with 1-4 R^{A};
each R^{A} is independently halo, hydroxyl, —CN, C₁₋₆ alkenyl, C₂₋₆ alkynyl, C₃₋₈ cycloalkyl, C₆₋₁₀ aryl, 4- to 10-membered heterocycloalkyl, 5- to 10-membered heteroaryl, —NH₂, —NH(C₁₋₆ alkyl), —N(C₁₋₆ alkyl)₂, —O(C₁₋₆ alkyl), or —O(C₃₋₈ cycloalkyl);
G¹ is a glycose unit having Formula II:

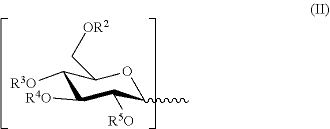

wherein:
   each R² and R³ is independently —H, -G¹, or

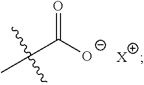

each R⁴ and R⁵ is independently —H or

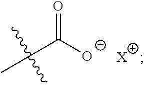

each X⁺ is independently a conjugate acid of a superbase; and
at least one instance of R², R³, R⁴, and R⁵ is

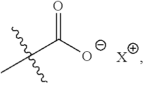

and
a degree of polymerization (DP) of the APG carbonate salt is from 1 to about 7.

Embodiment 2. The APG carbonate salt of embodiment 1, wherein each R³ is independently —H or -G¹.

Embodiment 3. The APG carbonate salt of embodiment 1, having Formula I-A:

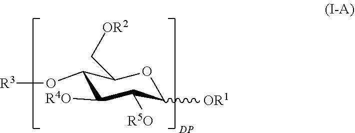

wherein:
each R², R³, R⁴, and R⁵ is independently —H or

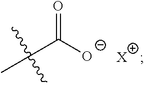

at least one instance of R², R⁴, R³, or R⁵ is

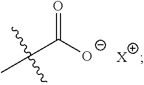

and
DP is from 1 to about 4.

Embodiment 4. The APG carbonate salt of any one of embodiments 1-3, wherein at least one instance of R² is

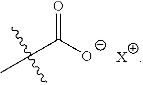

Embodiment 5. The APG carbonate salt of any one of embodiments 1-3, wherein one instance of R² is

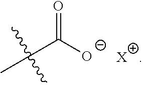

Embodiment 6. The APG carbonate salt of embodiment 1, having Formula I-B:

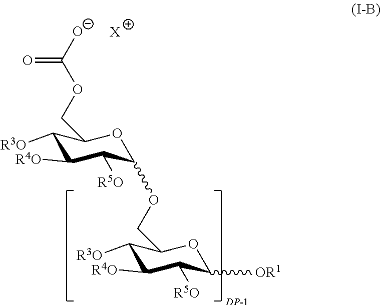

wherein (DP-1) is from 0 to about 3.

Embodiment 7. The APG carbonate salt of any one of embodiments 1-6, wherein each R³, R⁴, and R⁵ is —H.

Embodiment 8. The APG carbonate salt of any one of embodiments 1-7, wherein R¹ is C₆₋₁₈ alkyl.

Embodiment 9. The APG carbonate salt of any one of embodiments 1-7, wherein R¹ is C₈₋₁₆ alkyl.

Embodiment 10. The APG carbonate salt of any one of embodiments 1-9, wherein each X⁺ is independently a conjugate acid of an organic superbase.

Embodiment 11. The APG carbonate salt of any one of embodiments 1-9, wherein each X⁺ is independently a conjugate acid of 1,8-Diazabicyclo[5.4.0]undec-7-ene (DBU), 1,5,7-Triazabicyclo[4.4.0]dec-5-ene (TBD), 1,4-Diazabicyclo[2.2.2]octane (DABCO), 1,1,3,3-Tetramethylguanidine (TMG), 1,5-Diazabicyclo[4.3.0]-5-nonene (DBN), or 7-Methyl-1,5,7-triazabicyclo[4.4.0]dec-5-ene (MTBD).

Embodiment 12. The APG carbonate salt of any one of embodiments 1-9, wherein each X⁺ is:

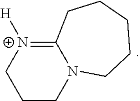 [SMILES: C1CCC2=[NH+]CCCN2CC1]

Embodiment 13. The APG carbonate salt of any one of embodiments 1-12, wherein DP is from 1 to about 2.

Embodiment 14. The surfactant composition of embodiment 13, wherein DP is from about 1.1 to about 1.5.

Embodiment 15. A method of forming an APG carbonate salt, comprising reacting an APG with CO₂ in the presence of a superbase, in a solvent.

Embodiment 16. The method of embodiment 15, comprising reacting an APG of Formula III:

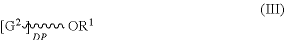

with CO₂, wherein:
R¹ is C₄₋₂₄ alkyl optionally substituted with 1-4 R^{A};
each R^{A} is independently halo, hydroxyl, —CN, C₁₋₆ alkenyl, C₂₋₆ alkynyl, C₃₋₈ cycloalkyl, C₆₋₁₀ aryl, 4- to 10-membered heterocycloalkyl, 5- to 10-membered heteroaryl, —NH₂, —NH(C₁₋₆ alkyl), —N(C₁₋₆ alkyl)₂, —O(C₁₋₆ alkyl), or —O(C₃₋₈ cycloalkyl);
G² is a glycose unit having Formula IV:

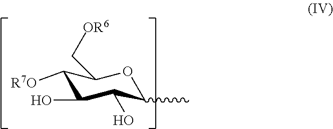

wherein each R⁶ and R⁷ is independently —H or -G², and
a degree of polymerization (DP) of the APG is from 1 to about 7.

Embodiment 17. The method of embodiment 15 or embodiment 16, wherein the superbase comprises an organic superbase.

Embodiment 18. The method of embodiment 15 or embodiment 16, wherein the superbase comprises DBU.

Embodiment 19. The method of any one of embodiments 15-18, wherein the solvent comprises dimethyl sulfoxide (DMSO).

Embodiment 20. An APG carbonate salt, prepared by the method of any one of embodiments 15-19.

Embodiment 21. A method of reducing a viscosity of a heavy oil, the method comprising adding an aqueous solution comprising the APG carbonate salt of any one of embodiments 1-14 and 20 to the heavy oil.

Embodiment 22. A method of displacing heavy oil, the method comprising injecting, through a wellbore into a subterranean formation comprising the heavy oil, an aqueous solution comprising the APG carbonate salt of any one of embodiments 1-14 and 20.

Embodiment 23. The method of embodiment 21 or embodiment 22, wherein the aqueous solution comprises about 0.01 wt % to about 5 wt % of the APG carbonate salt.

Embodiment 24. The method of any one of embodiments 21-23, wherein the aqueous solution comprises about 10,000 mg/L to about 100,000 mg/L of ions.

Other implementations are also within the scope of the following claims.

1. An alkyl polyglycoside (APG) carbonate salt of Formula I: 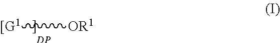
wherein:
R¹ is C₄₋₂₄ alkyl optionally substituted with 1-4 R^{A};
each R^{A} is independently halo, hydroxyl, —CN, C₁₋₆ alkenyl, C₂₋₆ alkynyl, C₃₋₈ cycloalkyl, C₆₋₁₀ aryl, 4- to 10-membered heterocycloalkyl, 5- to 10-membered heteroaryl, —NH₂, —NH(C₁₋₆ alkyl), —N(C₁₋₆ alkyl)₂, —O(C₁₋₆ alkyl), or —O(C₃₋₈ cycloalkyl);
G¹ is a glycose unit having Formula II: 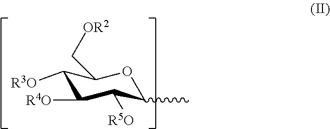
wherein:
each R² and R³ is independently —H, -G¹, or 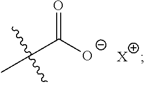
each R⁴ and R⁵ is independently —H or 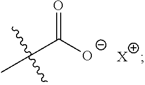
each X⁺ is independently a conjugate acid of a superbase; and
at least one instance of R², R³, R⁴, and R⁵ is 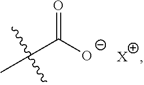
and
a degree of polymerization (DP) of the APG carbonate salt is from 1 to about 7.

2. The APG carbonate salt of claim 1, wherein each R³ is independently —H or -G¹.

3. The APG carbonate salt of claim 1, having Formula I-A: 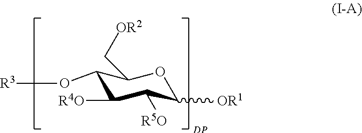
wherein:
each R², R³, R⁴, and R⁵ is independently —H or 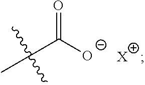
at least one instance of R², R⁴, R³, or R⁵ is 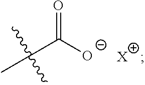
and
DP is from 1 to about 4.

4. The APG carbonate salt of claim 1, wherein at least one instance of R² is O

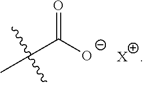

5. The APG carbonate salt of claim 1, wherein one instance of R² is

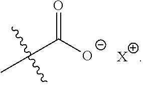

6. The APG carbonate salt of claim 1, having Formula I-B: 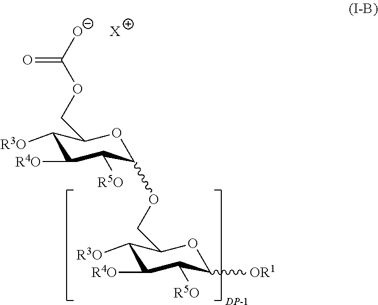
wherein (DP-1) is from 0 to about 3.

7. The APG carbonate salt of claim 1, wherein each R³, R⁴, and R⁵ is —H.

8. The APG carbonate salt of claim 1, wherein R¹ is C₆₋₁₈ alkyl.

9. The APG carbonate salt of claim 1, wherein R¹ is C₈₋₁₆ alkyl.

10. The APG carbonate salt of claim 1, wherein each X⁺ is independently a conjugate acid of an organic superbase.

11. The APG carbonate salt of claim 1, wherein each X⁺ is independently a conjugate acid of 1,8-Diazabicyclo[5.4.0]undec-7-ene (DBU), 1,5,7-Triazabicyclo[4.4.0]dec-5-ene (TBD), 1,4-Diazabicyclo[2.2.2]octane (DABCO), 1,1,3,3-Tetramethylguanidine (TMG), 1,5-Diazabicyclo[4.3.0]-5-nonene (DBN), or 7-Methyl-1,5,7-triazabicyclo[4.4.0]dec-5-ene (MTBD).

12. The APG carbonate salt of claim 1, wherein each X⁺ is:

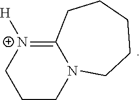 [SMILES: C1CCC2=[NH+]CCCN2CC1]

13. The APG carbonate salt of claim 1, wherein DP is from 1 to about 2.

14. The surfactant composition of claim 13, wherein DP is from about 1.1 to about 1.5.

15. A method of forming an APG carbonate salt, comprising reacting an APG with CO₂ in the presence of a superbase, in a solvent.

16. The method of claim 15, comprising reacting an APG of Formula III: 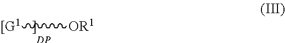
with CO₂, wherein:
R¹ is C₄₋₂₄ alkyl optionally substituted with 1-4 R^{A};
each R^{A} is independently halo, hydroxyl, —CN, C₁₋₆ alkenyl, C₂₋₆ alkynyl, C₃₋₈ cycloalkyl, C₆₋₁₀ aryl, 4- to 10-membered heterocycloalkyl, 5- to 10-membered heteroaryl, —NH₂, —NH(C₁₋₆ alkyl), —N(C₁₋₆ alkyl)₂, —O(C₁₋₆ alkyl), or —O(C₃₋₈ cycloalkyl);
G² is a glycose unit having Formula IV: 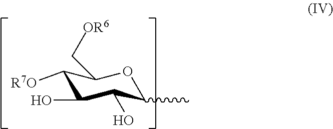
wherein each R⁶ and R⁷ is independently —H or -G², and
a degree of polymerization (DP) of the APG is from 1 to about 7.

17. The method of claim 15, wherein the superbase comprises an organic superbase.

18. The method of claim 15, wherein the superbase comprises DBU.

19. The method of claim 15, wherein the solvent comprises dimethyl sulfoxide (DMSO).

20. An APG carbonate salt, prepared by the method of claim 15.

21. A method of reducing a viscosity of a heavy oil, the method comprising adding an aqueous solution comprising the APG carbonate salt of claim 1 to the heavy oil.

22. A method of displacing heavy oil, the method comprising injecting, through a wellbore into a subterranean formation comprising the heavy oil, an aqueous solution comprising the APG carbonate salt of claim 1.

23. The method of claim 21, wherein the aqueous solution comprises about 0.01 wt % to about 5 wt % of the APG carbonate salt.

24. The method of claim 21, wherein the aqueous solution comprises about 10,000 mg/L to about 100,000 mg/L of ions.
